(19) United States
(12) Patent Application Publication
(10) Pub. No.: US 2006/0064113 A1
(43) Pub. Date: Mar. 23, 2006

(54) Endoscopic mucosal resection method and associated instrument

(76) Inventor: Naomi L. Nakao, New York, NY (US)

Correspondence Address: COLEMAN SUDOL SAPONE, P.C., 714 COLORADO AVENUE, BRIDGE PORT, CT, 06605-1601, US

(21) Appl. No.: 11/201,579

(22) Filed: Aug. 11, 2005

Related U.S. Application Data
(60) Provisional application No. 60/611,260, filed on Sep. 17, 2004.

(51) Int. Cl. A61B 17/26

(52) U.S. Cl. 606/113

(57) ABSTRACT

An endoscopic tissue resection device and related method is used in conjunction with a flexible or rigid endoscope. Tissue is resected by shaving thin layers of tissue for diagnostic and therapeutic purposes.

### CROSS-REFERENCE TO RELATED APPLICATION

This application claims the benefit of U.S. Provisional Patent Application No. 60/611,260 filed Sep. 17, 2004.

### BACKGROUND OF THE INVENTION

This invention relates to endoscopic medical procedures and more particularly to endoscopic mucosal resection procedures. This invention also relates to an endoscopic instrument or assembly utilizable in performing an endoscopic mucosal resection procedure.

The precancerous nature of high-grade dysplasia and the difficulty in detection of invasive carcinoma by endoscopy make esophagectomy and ablative therapy important considerations to treating those patients with this serious condition. The gold standard treatment for early esophageal cancer and high grade dysplasia is esophagectomy, the surgical removal of the diseased segment of the esophagus. This is an effective but drastic treatment and presents significant complications and lifestyle problems for the patient. Many patients are poor surgical candidates for this difficult surgery.

Endoscopic mucosal resection (EMR), the removal of mucosal tissue by use of a snare, is a therapeutic alternative and has become a standard treatment for patients with Barrett's Esophagus. This technique preserves the patient's esophagus while resecting the mucosa that is affected by this disease. A second method is tissue ablation with heat therapy. EMR is superior to tissue destruction because it permits pathologic evaluation of the resected specimen. Current endoscopic mucosal resection techniques for the treatment of esophageal cancer include strip biopsy, double snare polypectomy, with the combined use of saline and epinephrine injection. EMR may be curative if the primary tumor or dysplastic tissue is removed completely.

Another area where EMR may be used is for removal of large sessile polyps in the GI tract, primarily the colon. The malignant transformation potential of colorectal adenomatous polyps is well documented. Colonoscopic polypectomy is widely practiced in order to prevent the development of colon cancer. Sessile polyps are premalignant lesions that lay flatly on the mucosal surface of the colon wall. These lesions, in contrast to pedunculated polyps, are devoid of a stalk, and are broad based. The colon wall is composed of several layers: the mucosa (the surface layer), the submucosa, the muscularis (muscle layer), and the serosa (connective tissue layer). The thickness of the entire wall is 5 mm. When a cautery snare is used to remove a larger sessile lesion, it may catch part of the muscularis layer Cutting through the muscle layer causes a colonic perforation.

Devices currently used for EMR procedures are polypectomy snares and a variety of devices to assist in the use of these snares. For resection of dysplastic tissue in the esophagus the technique involves using 2 snares, one to hold up the targeted tissue and the other to sever that tissue. The use of saline solutions for injection beneath the target tissue is a common practice for the purpose of raising the tissue and creating a buffer layer. This process is called saline assisted polypectomy (SAP).

In the case of sessile colonic polyps, SAP is standard medical practice. The raised polyp is then severed with a polypectomy snare, often in several segments (segmental resection) depending on the size and location of the polyp.

The depth of the cut that occurs using the snare cautery device to remove dysplastic mucosal tissue is critical. As discussed above, if the cut is too deep, injuring the muscularis layer, a perforation may occur. Conversely, a cut too shallow may not remove enough of the affected tissue and therefore may require additional procedures, or worse, result in the development of metastatic cancer. Similar complications may occur during the removal of sessile colonic polyps. The colonic wall is approximately the same thickness as the esophageal wall, namely 5 mm. A perforation as a result of cutting into the muscularis layer will cause a colonic perforation, while a lesion that is not completely removed, either due to insufficient depth or breath, will result in recurrence of the dysplastic tissue. Repeated resections after a certain interval are recommended if the margin of resection achieved during the procedure is too close to the tumor. More than 2 mm of cancer clearance is required. The complications resulting from EMR as performed with today's devices and methods include perforation, bleeding, and strictures that occur from scar formation resulting from EMR procedures.

Ablation techniques rely on chemicals which, when combined with heat or freezing, destroy dysplastic tissue. Adverse reactions include destruction of the healthy tissue surrounding the lesion, allergic reactions to the chemicals and sensitivity to sun-light. Furthermore, all ablative techniques destroy the tissue and prevent adequate pathologic examination of the specimen.

### OBJECTS OF THE INVENTION

An object of the present invention is to provide a method for resecting dysplastic tissue masses disposed along internal organ walls.

It is a more particular object of the present invention to provide an instrument that will enable accurate removal of tissue that lies flatly on the mucosal wall of the gastrointestinal tract.

It is another more particular object of the present invention to provide such a method and/or instrument that reduces the likelihood of organ perforation.

It is another object of the present invention to provide such a method that is minimally invasive.

It is even a more particular object of the present invention to provide an instrument and accompanying method that enables control of the depth and breadth of resection.

A further object of the present invention is to provide such a method that is carried out endoscopically.

It is a particular object of the present invention to provide an instrument that may be used in conjunction with a flexible endoscope, whereby the instrument's end effector is larger than the working channel of the endoscope.

These and other objects of the invention will be apparent from the drawings and descriptions herein. Although every object of the invention is believed to be achieved by at least one embodiment of the invention, there is not necessarily any single embodiment that achieves all of the objects of the invention.

### SUMMARY OF THE INVENTION

A medical device comprises, in accordance with the present invention, at least one elongate instrument shaft insertable through a working channel of an endoscope, a holder member provided at a distal end of the instrument shaft, and a cutting wire element connected to the holder member. The wire element extends between spaced points of the holder member in a use configuration of the holder member and the wire element.

In several embodiments of the invention, the cutting wire is made of electrically conductive material operatively connectable to a source of electrical current, thereby enabling a cauterization of organic tissues during a cutting operation.

Typically, the holder member has a Y- or V-shaped configuration in the use configuration, the holder member having a pair of arms extending at an angle relative to one another. The wire element extends in a straight line from one arm of the holder member to another arm thereof. The arms of the holder member may be pivotably connected to one another and disposed in an insertion configuration inside the working channel of the endoscope. After insertion of distal end portion of the endoscope into a patient, the operative tip of the instrument is ejected from the endoscope working channel. The operative tip is then reconfigured from the insertion configuration to the use configuration.

The insertion configuration of the holder member may be a collapsed configuration, in which case the change in conformation of the operative tip involves an opening of the holder member, a spreading of two pivotably interconnected arms, and the stretching of the wire element from a loose or flaccid loop to a straight and taut line.

Alternatively, the insertion configuration of the holder member may be a straight line configuration having a pair of joints, bend points, or articulations. Upon the ejection of the holder member from the working channel of the endoscope (or from a tubular introducer sheath itself slidably inserted inside the working channel), the holder member is folded at the joints, bend points, or articulations to form a triangular or V-shaped use configuration. This conformation change may be effectuated by pulling on the wire element, which is fixedly connected to the tip of the holder element, and on a second wire which is connected to a distal-most joint, bend point, or articulation of the holder member.

In either of the above-described embodiments of the holder member, the arms are pivotably connected to one another. In the one embodiment, the arms are each connected at a proximal end to the instrument shaft. The arms may be spring biased towards an opened configuration. In the other embodiment, the arms are connected in series to one another and to the instrument shaft, the one arm being connected at one end to the instrument shaft and at an opposite end to the other arm.

Where the holder member comprises a pair of jaws pivotably connected to one another and to the distal end of the instrument shaft, the wire element originates from a proximal end of the instrument shaft, extends along the instrument shaft and one of the arms, and forms a straight line from the one arm to the other arm in the use configuration.

In yet another embodiment of the present invention, the arms of the holder member are rigid elements fixed to one another exemplarily in a Y- or V-shaped configuration. In that case, the holder member arms and the wire element are disposable in a plane oriented perpendicularly to the instrument shaft, at least during an insertion or deployment procedure. More particularly, the holder member and the wire element are disposable along a distal end face of an endoscope member and inserted into the patient while riding on the front or distal end face of the endoscope insertion member. The holder member may be removably connectable to the end of the instrument shaft. In that case, before initiation of the endoscopic procedure, and in preparation thereof, the instrument shaft is inserted into the working channel of the endoscope from the proximal end. The holder member is subsequently screwed onto or otherwise connected to the distal end of the instrument shaft as it protrudes from the distal end of the endoscope working channel. After formation of this connection, the instrument shaft is pulled in the proximal direction until the holder member and the wire element lay snugly against the leading or distal end face of the endoscope insertion member. The endoscope insertion member is inserted into the patient with the operative tip (holder member and cauterizing wire element) engaging or touching the distal end face of the endoscope. The wire element preferably takes a substantially semi-rigid arcuate form that fits around the periphery of the endoscope end face. The two arms of the holder member are positioned such that the visualization optics, air channel, lens, and biopsy channels of the endoscope are not obstructed. Similarly, the wire element's positioning around the periphery is also such as not to interfere with these essential elements of the endoscope's distal end.

A medical method in accordance with the present invention utilizes a medical instrument including an elongate wire element coupled to a holder member. At least a portion of the instrument including the wire element and the holder member is introduced into a patient via an endoscope Thereafter the wire element is placed into engagement with a patient's organic tissue at a surgical site. The wire element is moved into and along the tissue to remove a thin layer or web of the tissue.

Optionally, in certain preferred embodiments of the present invention, the wire element is made of electrically conductive material and is connectable to a source of electric current. During the motion of the wire element in such embodiments, electrical current is conducted into the wire element to facilitate a cutting and cauterizing of the tissue at the surgical site.

The wire element may extend between two points on the holder member. The moving of the wire element into and along the tissue may then include pulling the holder member from outside the patient to draw the wire element towards a distal end face of the endoscope. More particularly, movement of the wire element may be effectuated by moving the instrument shaft and holder assembly relative to the endoscope or, alternatively, by holding the cutting wire and holder element stationary relative to the endoscope, and manipulating the endoscope to guide the cutting and cauterizing action.

The holder member may include a pair of arms pivotably connected to one another. In that case, the method may further comprise opening the arms from a collapsed configuration to an opened use configuration upon an ejection of the holder member from a distal end of the working channel. The arms may be opened by pivoting the arms relative to one another.

The holder member may include a pair of arms connected in series to one another and to the instrument shaft, one of the arms being connected at one end to the instrument shaft and at an opposite end to another of the arms. In that case, the method may further comprise bending or folding the arms relative to one another to form a use configuration upon an ejection of the holder member in a straightened configuration from a distal end of the working channel. The bending or folding of the arms may include a pivoting of the arms relative to one another.

In another embodiment of the present invention, the method may comprise disposing the holder member and the wire element along a distal end face of an insertion member of the endoscope. The introducing of the wire element and the holder member into the patient may include inserting the insertion member into the patient with the wire element and the holder member disposed along the distal end face of the endoscope insertion member. In this embodiment of the invention, a shaft of the medical instrument may be inserted into the working channel of the endoscope and then the holder member attached to the instrument shaft. The disposing of the holder member and the wire element along the distal end face is performed subsequently to the attaching of the holder member to the instrument shaft.

### BRIEF DESCRIPTION OF THE DRAWINGS

FIG. 1 is a perspective view of a first embodiment of a cutting and cauterizing instrument as it emerges from the distal end of an endoscope, in accordance with the present invention.

FIG. 2 is a perspective view of the cutting and cauterizing instrument of FIG. 1, upon a manipulation of push bars or wires by actuation of the handle mechanism.

FIG. 3 is a perspective view of the cutting and cauterizing instrument of FIGS. 1 and 2 in a fully open position ready for tissue resection.

FIG. 4 is a perspective view of the cutting and cauterizing instrument of FIGS. 1-3 slicing a section of tissue.

FIG. 5 is a perspective view of a handle mechanism of the cutting and cauterizing instrument of FIGS. 1-4.

FIG. 6 is a perspective view of an alternative embodiment of a cutting and cauterizing instrument pursuant to the present invention, showing an operative tip of the instrument in a collapsed configuration inside a tubular introducer or deployment member.

FIG. 7 is a perspective view of the cutting and cauterizing instrument of FIG. 6 wherein a push bar or wire is advanced distally and two holder rods or arms of the operative tip begin to spread apart into an open configuration.

FIG. 8 is a perspective view of the cutting and cauterizing instrument of FIGS. 6 and 7, showing the operative tip in the fully open configuration ready for tissue resection.

FIG. 9 is a perspective view of the cutting and cauterizing instrument of FIGS. 6-8, showing the instrument being used to slice a section of tissue along a wall of an internal organ.

FIG. 10 is a perspective view of the handle mechanism for the embodiment of FIG. 6.

FIG. 11 illustrates an alternative embodiment of a cutting and cauterizing instrument, in accordance with the present invention.

FIG. 12 is a blown up view of an arch structure of the instrument or device of FIG. 11.

FIG. 13 is a schematic perspective view of a distal working end of an alternative embodiment of the cutting and cauterizing instrument, in accordance with the present invention.

FIG. 14 is a schematic longitudinal cross-sectional view of the device of FIG. 13.

FIG. 15 is a schematic perspective view of a further alternative embodiment of a cutting and cauterizing instrument, in accordance with the present invention.

FIG. 16 is a schematic perspective view of a scissor-type handle mechanism alternatively utilizable with the cutting and cauterizing instrument of FIG. 15.

FIG. 17 is a schematic perspective view of a distal working end of the cutting and cauterizing instrument shown in FIG. 15, with jaws in an open configuration.

FIG. 18 is a schematic side elevational view, partially in section, of the cutting and cauterizing instrument of FIG. 15, with jaws in a partially closed configuration.

FIG. 19 is a schematic side elevational view, partially in section, of the cutting and cauterizing instrument of FIG. 15, with jaws in a completely closed configuration.

FIG. 20 is a side elevational view, partly in section, of another alternative embodiment of a cutting and cauterizing instrument, with jaws in an open use configuration ready for cutting.

FIG. 21 is a schematic perspective view of the cutting and cauterizing instrument of FIG. 20, with jaws in the open use configuration.

FIG. 22 is a schematic cutaway or longitudinal cross-sectional view of the cutting and cauterizing instrument of FIGS. 20 and 21, in a closed, pre-deployment configuration.

FIG. 23 is a schematic perspective view of the cutting and cauterizing instrument of FIGS. 20-22, in the closed, pre-deployment configuration.

FIG. 24 is a perspective view of a jaw or arm element of a wire holder member utilizable as a modification of the cutting and cauterizing instrument of FIGS. 20-24.

FIG. 25 is a longitudinal cross-sectional view of the jaw or arm element shown in FIG. 24.

FIG. 26 is a front end elevational view of the jaw or arm element of FIGS. 24 and 25.

FIG. 27 is a schematic elevational view of a further modification of the cutting and cauterizing instrument of FIGS. 20-23.

FIG. 28 is a schematic side elevational view, on a reduced scale, of another embodiment of an endoscopic cutting and cauterizing instrument in accordance with the present invention, showing an operative tip in a folded use configuration.

FIG. 29 is a schematic perspective view, on a larger scale, of a distal end portion of the endoscopic cutting and cauterizing instrument of FIG. 28, showing the distal end portion, including a wire element and a holder member, in a straightened insertion configuration.

FIG. 30 is a schematic perspective view, on an even larger scale, of the distal end portion of the endoscopic cutting and cauterizing instrument of FIGS. 28 and 29, showing the distal end portion in a partially folded configuration.

FIG. 31 is a schematic perspective view, on an enlarged scale, of the distal end portion of the endoscopic cutting and cauterizing instrument of FIGS. 28-30, showing the distal end portion in the completely folded, use configuration of FIG. 28.

FIG. 32 is a schematic perspective view of the distal end portion of the endoscopic cutting and cauterizing instrument of FIGS. 28-31, showing a stage in the use of the instrument to plane or shave a layer of tissue from inside a tubular organ.

FIG. 33 is a schematic perspective view of an endoscope assembly including another embodiment of a cutting and cauterizing instrument in accordance with the present invention.

FIGS. 34A through 34D are schematic perspective views of a distal end portion of the endoscope of FIG. 33, showing successive steps in forming an insertion configuration of the cutting and cauterizing instrument together with the endoscope.

FIGS. 35A through 35C are schematic perspective views of the distal end portion of the endoscope of FIGS. 33 and 34A-34D, showing successive steps in the use of the instrument assembly in an endoscopic resection procedure, in accordance with the present invention.

FIGS. 36A and 36B show successive steps in utilization of a modification of the cutting and cauterizing instrument assembly of FIGS. 33, 34A-34D, and 35A-35C.

FIG. 37A is a schematic side elevational view, partially in cross-section, of a modification of the cutting and cauterizing instrument assembly of FIGS. 36A and 36B, showing a transverse or angled configuration of the assembly.

FIG. 37B is a schematic side elevational view of the cutting and cauterizing instrument assembly of FIG. 37A, showing a straightened configuration of the assembly.

FIG. 37C is a schematic end elevational view of the cutting and cauterizing instrument assembly of FIG. 37A, taken from the left side in FIG. 37A.

### DEFINITIONS

The present invention is directed to a medical or surgical procedure for removing an undesirable tissue mass located along the surface of a lumen of an internal organ such as the esophagus or the colon. Typically, multiple passes are made along the tissue mass by a shaving device as described herein, to ablate a series of web- or sheet-shaped portions of the undesirable tissue mass in a controlled fashion.

The following are definitions of some terms used in this disclosure.

The term “wire element” is used herein to denote a thin elongate cutting member that functions to ablate or otherwise cut organic tissues of a patient in a shaving procedure. Such a cutting element is preferably but not necessarily made of an electrically conductive material, generally a metal or alloy. In that case cutting and cauterizing is effectuated in large part by hear generated owing to the conduction of electrical current. Alternatively, the wire may cause cutting by freezing, or by slicing through tissue such as a cheese cutter would slice through cheese, simply by virtue of the wire's sharpness. A wire element as disclosed herein may be flexible or substantially rigid or semi-rigid. A semi-rigid wire element has some flexibility but has an inherent spring bias that tends to returns the wire to a preselected configuration, such as a circular arc. The wire element may be connected at spaced points to a holder member.

The term “holder member” is used herein to denote a support for a wire element. In some embodiments of the invention, an active portion of the wire element extends between two points of the holder member so as to be free to ablate and cauterize or otherwise resect abnormal tissue.

### DETAILED DESCRIPTION OF THE INVENTION

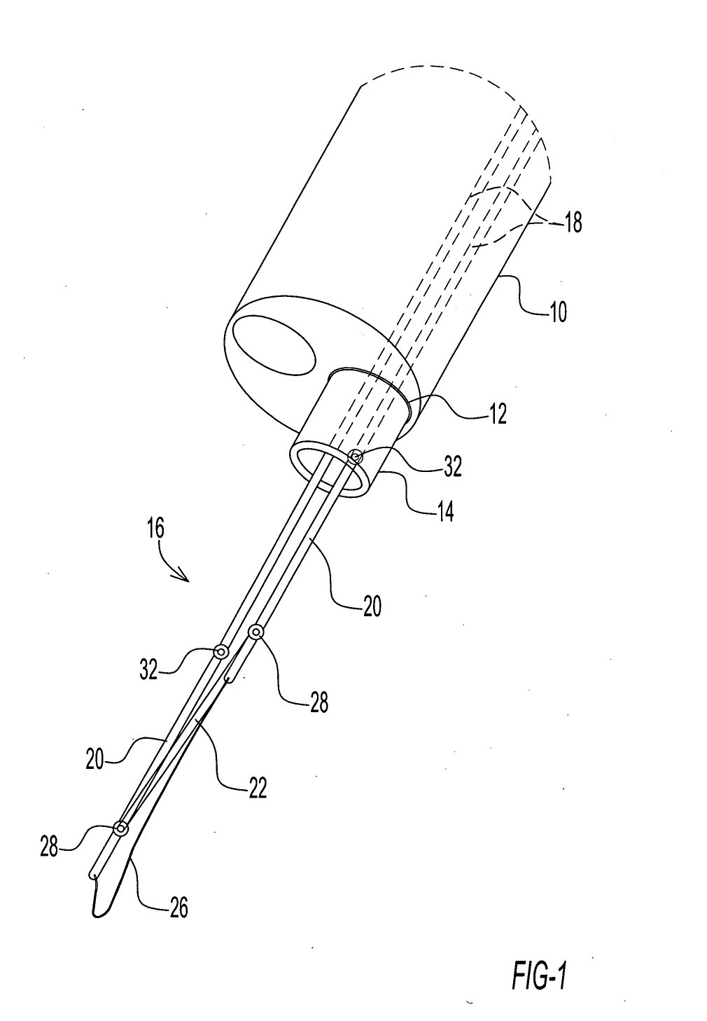

FIGS. 1-5 illustrate one embodiment of the invention that includes an electrocautery device capable of being passed through the working channel 12 of an endoscope 10. Referring to FIG. 1, an elongated tube 14 might be positioned in the channel 12 to emerge from the distal end of endoscope 10. The cutting device or assembly 16 is moved through the elongate tube 14 to emerge therefrom. The device 16 includes one or more push bars or wires 18 that are generally parallel to one another. The push bars or wires 18 are coupled to a corresponding pair of rigid or semi rigid electrically conductive rod elements or rods 20 and a crossbar element 22 that spans between the rod elements 20. Rod elements 20 and crossbar 22 sever as a holder for a cutting wire 26. Together, rod elements 20, crossbar 22 and wire 26 are an operative tip of the electrocautery device.

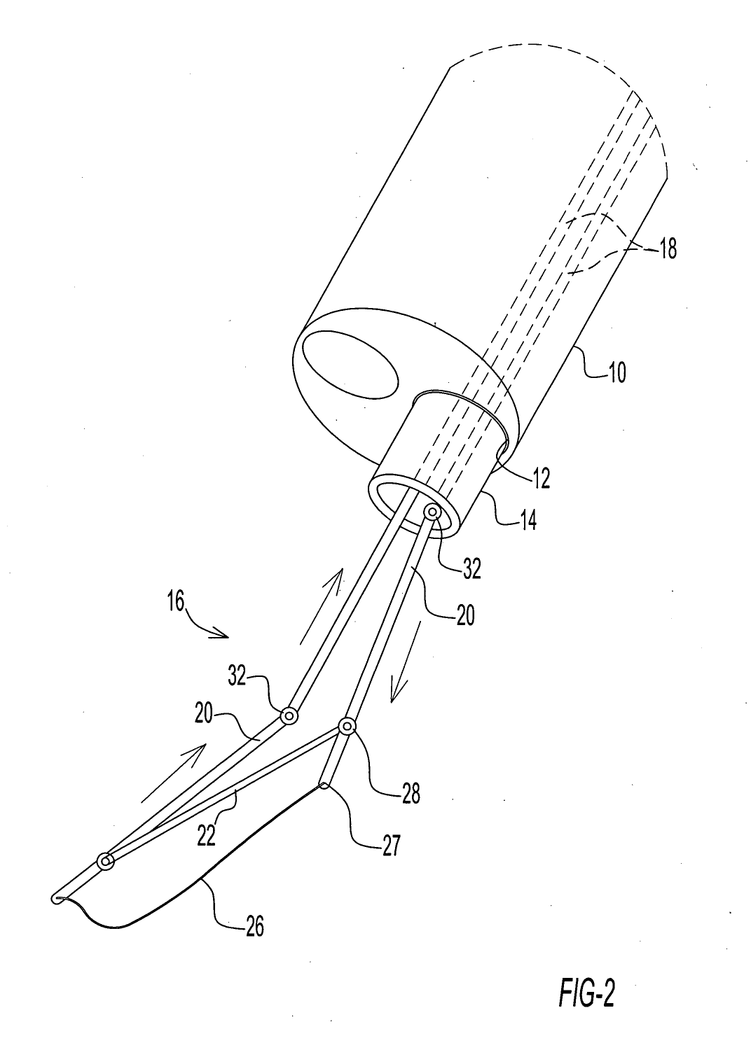

As further illustrated in FIG. 2, cutting wire 26 spans between the distal ends 27 of the rod elements 20. FIG. 1 shows the device 16 and the cutting wire 26 in a collapsed position for sliding through the channel 12 and tube 14. The crossbar element 22 and rod elements 20 are pivotally connected together by hinge/pivot elements or pivots 28. Similarly, the rod elements 20 are coupled to the push bars or wires 18 by pivots 32.

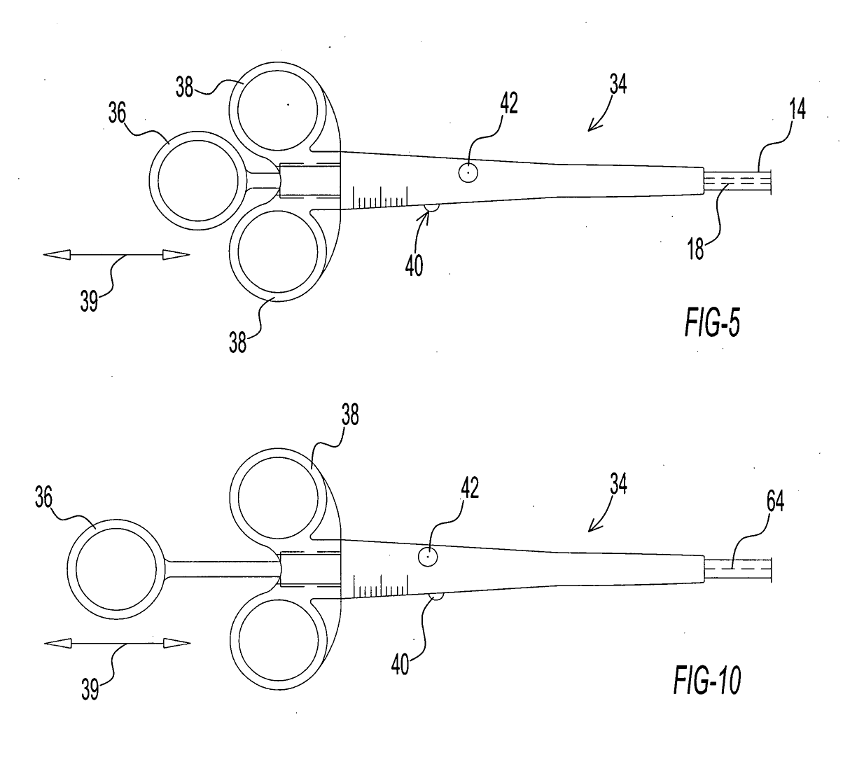

The device 16 has a handle mechanism 34 at the proximal end of the cutting device, as shown in FIG. 5, which is coupled to the push bars or wires 18. The handle mechanism 34 is distally connected to elongate tube 14 and includes a lockable thumb ring 36, slideable in perspective to finger rings 38, and connected to push bars 18. The thumb ring 36 is slideable as indicated by arrow 39. A lock 40 may be used to lock the thumb ring 36 with respect to the finger rings 38. The handle mechanism also includes an electrical connector 42.

The handle mechanism 34 may be connected at its proximal end, through the electrical connector 42, to an electro-surgical generator. The push bars or wires 18 may also conduct electric current to the cutting mechanism, such as to the cutting wire 26, so as to heat the cutting wire to provide a cauterizing effect when the wire is used to cut tissue as discussed below.

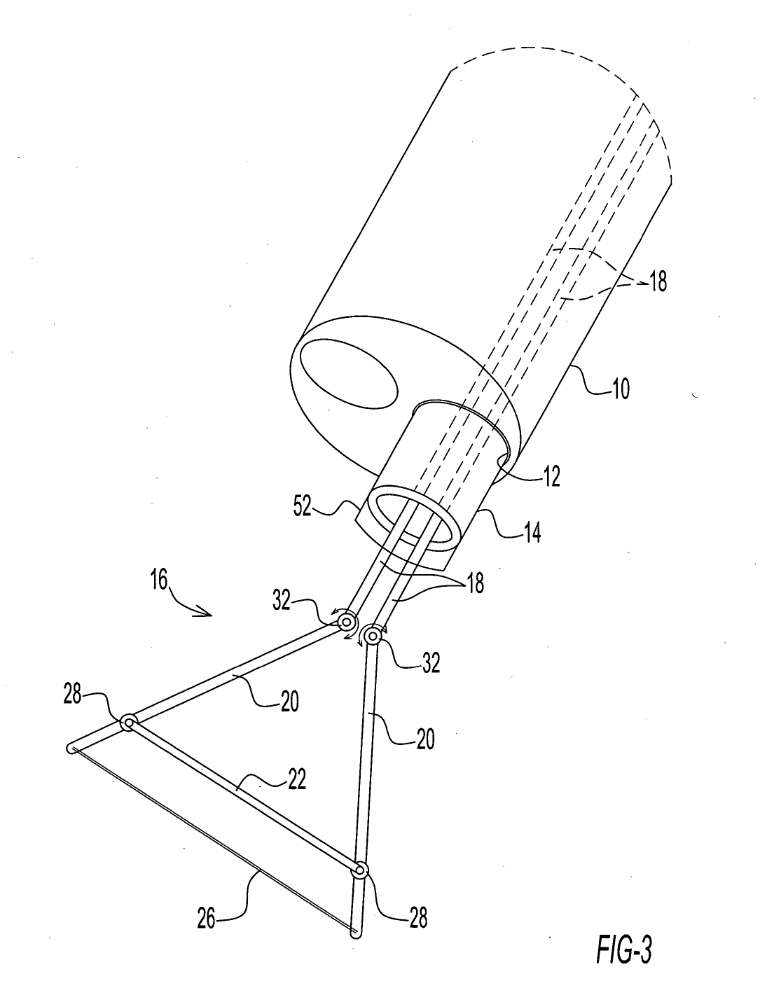

The distal end of the device 16, as shown in FIG. 1, is composed of elements that are capable of being collapsed into the distal segment of the elongate tube 14 while being deployed through the working channel 12 of the endoscope 10. The cutting mechanism includes the rigid or semi rigid elements 20, and 22 which, when fully deployed, form an “A” shape, as shown in FIG. 3, with the top of the “A” in slidable contact with the push bars or wires 18. With reference to FIG. 2, the device 16 is pushed from the end of tube 14, such as by motion of the thumb ring 36. The motion of the thumb ring with respect to the finger rings causes one push bar or wire 18 to advance distally, as shown in FIG. 2, and the other push bar or wire 18 to retract proximally. The push bars or wires 18 move the rod elements 20, which pivot with respect to the push bars or wires on pivots 32. This motion of the rod elements 20, in turn, causes the crossbar 22 to pivotally move on pivots 28 to span between the wire elements 20 and to cause the wire elements 20 to angle away from each other and to ultimately form the “A” shape as shown in FIG. 3. The cutting wire 26 is then stretched to span between the distal ends of the rod elements 20.

The crossbar 22 of the “A” is covered with an insulating heat shrink material and is connected to each leg (rod element 20) of the “A”. The bottom or most distal segments of the rod elements 20 forming the “A” are connected to the electrically conductive cutting wire, which may be a very thin stainless steel mono-filament. As shown in FIGS. 1-3, the device 16 is collapsed and contained in the endoscope 10. When the device 16 is passed through the endoscope working channel 12 it is ejected from the distal end of the endoscope 10 and positioned near the tissue targeted for resection. As discussed above, the handle mechanism is pushed in the distal direction causing the push bars or wires to slide forward and open the cutting device to a use configuration by pushing the slidable connected rods 20 of the “A” down and the hinge connected crossbar 2 out forcing the lower portion of the “A” apart and the cutting wire to straighten and become firm. In that position, the push bars or wires 18 are aligned at their proximal ends, crossbar 22 is generally perpendicular to push bars or wires 18 and the cutting wire 26 is stretched taut and generally parallel to crossbar 22. The rod elements 20 are angularly connected to push bars or wires 18, crossbar 22 and cutting wire 26. The cutting wire 26 may be guided in the depth of cut by the crossbar 22.

In the position as shown in FIG. 3, the handle mechanism is locked into this position maintaining consistent tension on the cutting wire. The distance between the crossbar and cutting wire is predetermined and maybe set at various heights, such as at approximately 3 mm for example.

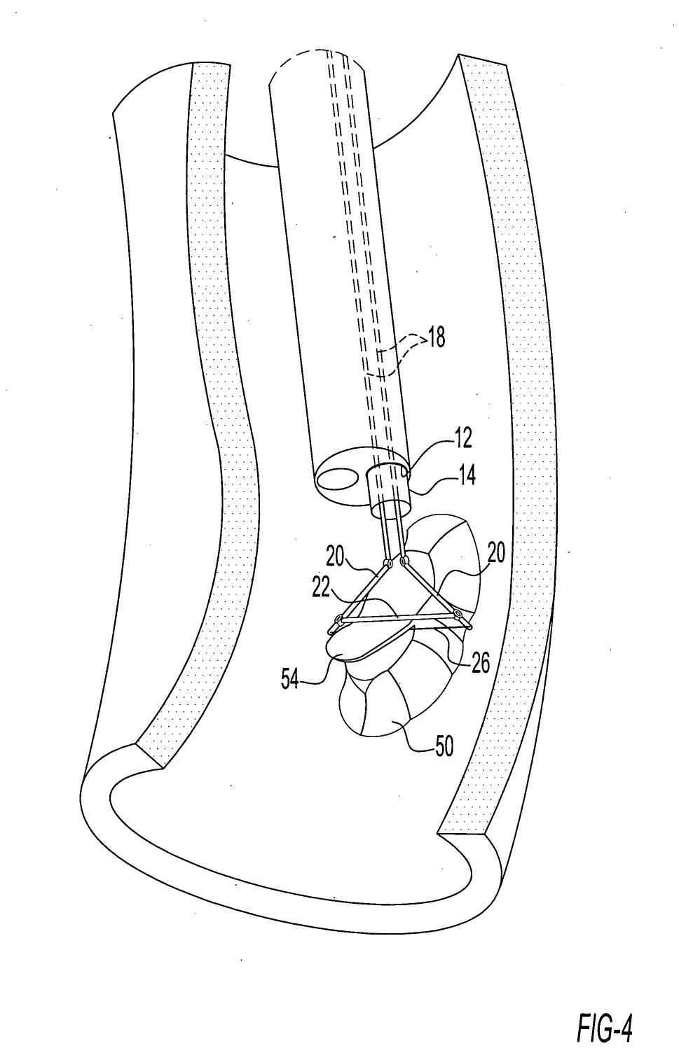

FIG. 4 depicts the cutting procedure in progress. The cutting wire 26 is placed slightly beyond the targeted tissue and cautery is applied as the heated cutting wire is drawn across a lesion 50 or other tissue. This action causes thin slicing through the tissue, such as a lesion or tumor, in the same fashion as a cheese cutter slicing through cheese. The preset distance of the cross bar 22 with respect to the cutting wire 26 prevents the cut from penetrating too deeply into the tissue. In order for the triangle or “A” shape structure to remain stationary and not rotate on its axis, it may be desirable to use a collar 52 or other structure (see FIG. 3) at the distal end of the tube 14 so that the triangle remains positioned in a stable manner. The severed tissue 54 is then captured with a grasper or retrieval net and withdrawn for pathologic evaluation.

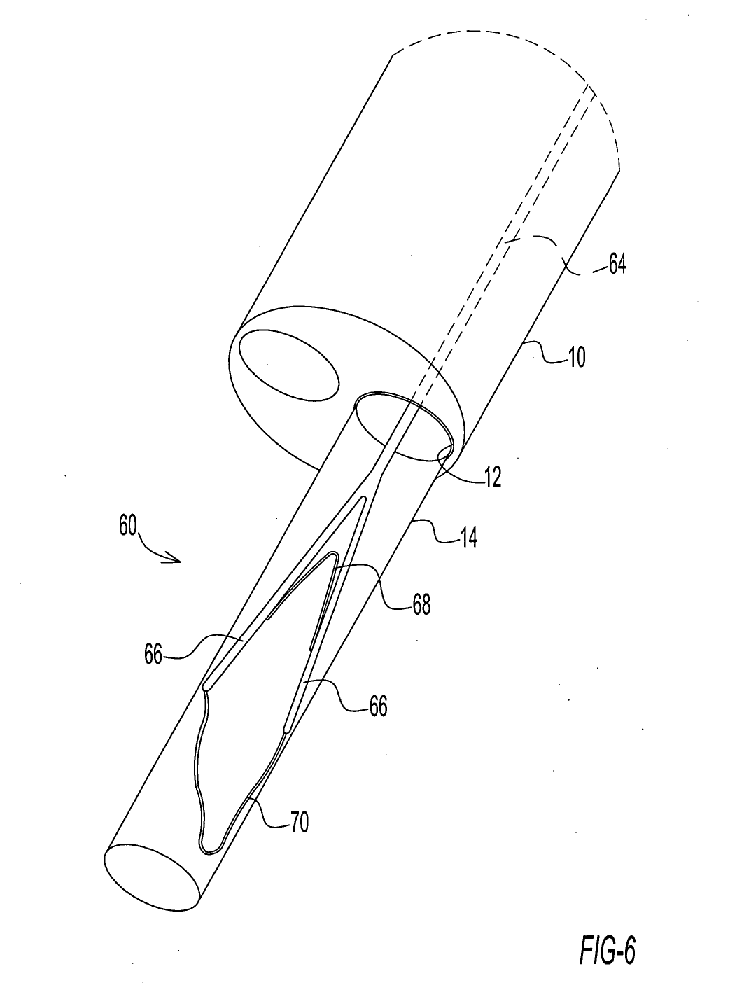

Another embodiment of the cutting device is shown in FIGS. 6-9. The cutting device 60 is an electrocautery device similar to device 16 that is capable of being passed through the working channel 12 of an endoscope 10 as shown in FIG. 6. The device 60 also uses an elongate tube 14, a handle mechanism 34 (FIG. 10), and a push rod 64, with a cutting mechanism at the distal end. The handle mechanism 34 may be connected at its proximal end to an electro-surgical generator through the connector 42. The push rod or wire 64 may also conduct electric current to the cutting mechanism as discussed above for cautery cuts of tissue. The distal end of the device 60 is composed of an embodiment that is capable of being collapsed into the distal segment of the elongate tube 14 (catheter) while being deployed through working channel 12 of the endoscope 10.

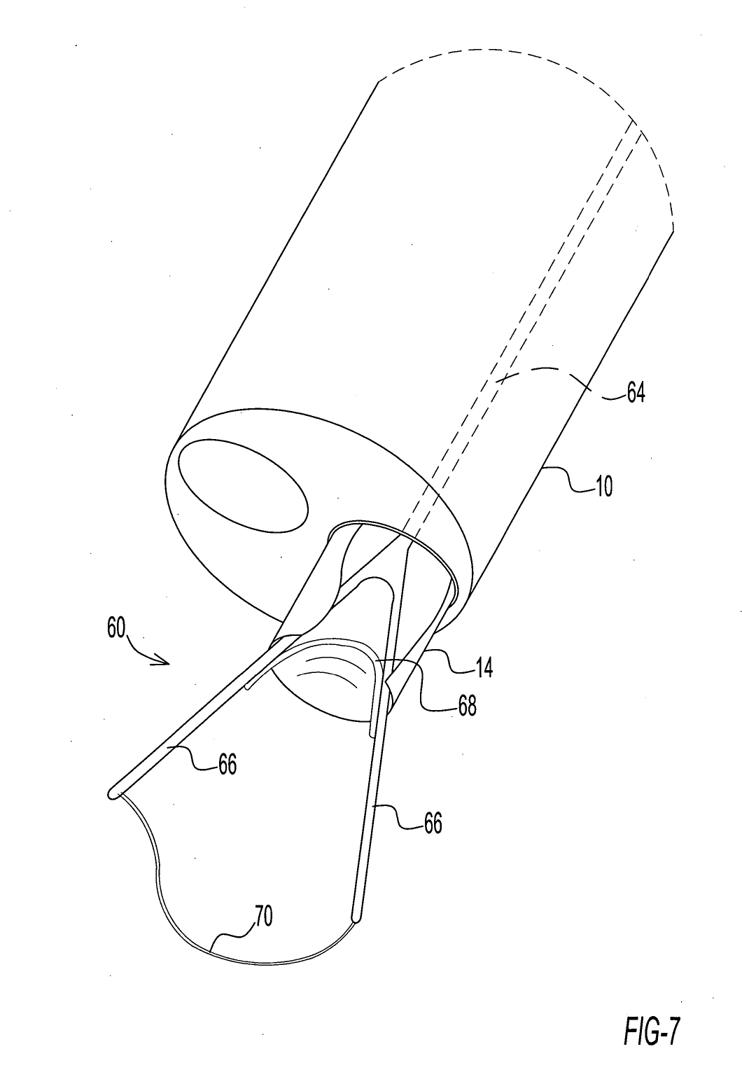

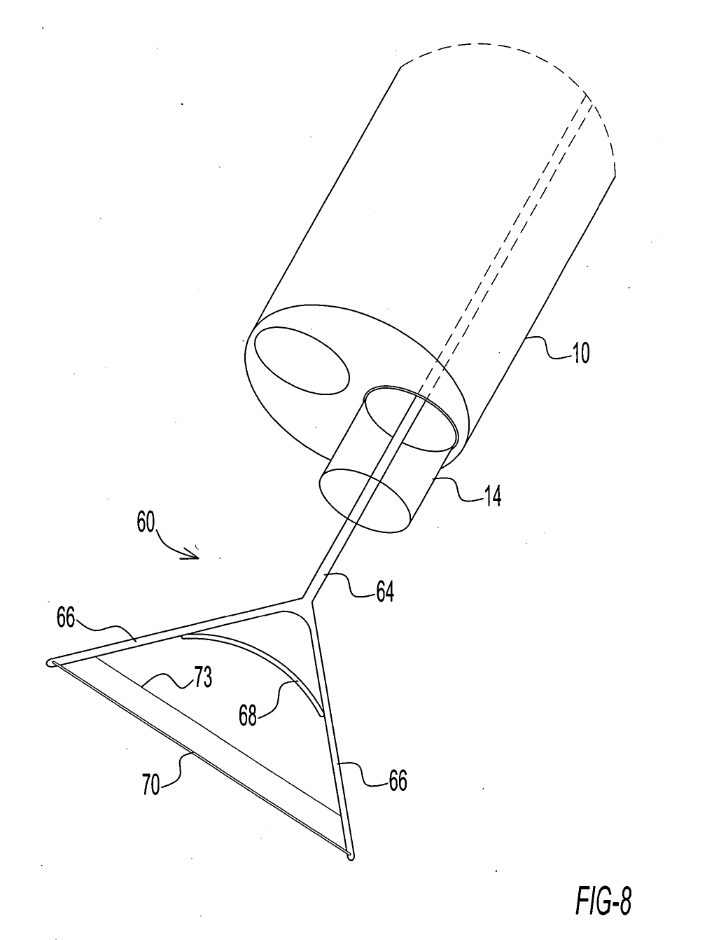

The cutting device or assembly includes two opposing rigid segments 66 that, when fully deployed, form an A shape with the top of the A in slidable contact with the push bar or wire. The rigid segments 66 are connected to a spring, such as in the form of a spring biased arch structure 68 or other spring structure. In the illustrated embodiment, the arch structure is n shaped, connected to the Λ shape of the opposing rigid segments 66 to drive the segments apart. The spring biased arch structure 68 is constructed in such a way as to bias the legs open as shown in FIGS. 7-8. A cutting wire 70 spans between the distal ends of the rigid segments 66. By actuation of the handle mechanism 34, the cutting wire 70, rigid segments 66, spring biased arch structure 68 and push rod 64 emerge from the distal end of elongate tube 14 as shown in FIG. 7. As spring biased arch structure 68 emerges it opens, pushing rigid segments 66 apart at the distal ends, thereby stretching the cutting wire 70 therebetween. The cutting wire 70 can be an electrically conductive wire, such as a very thin stainless steel mono-filament. With reference to FIG. 8, an A shape is formed by the device 60.

As depicted in FIG. 6, when the triangle is housed in the elongate tube 14 or catheter in the pre-deployment position, the rigid segments 66 are situated in a generally parallel position with one another, and the arch structure 68 and cutting wire 70 are collapsed inside the elongate tube 14. When the device is prepared for deployment, the handle is actuated causing the push-bar or wire to advance in the distal direction. This causes the two rigid triangle legs to be ejected from the catheter as demonstrated in FIG. 7. The spring biased arch structure 68 springs open, thereby separating the legs and straightening the distal cutting wire 70 at the base of the triangle (FIG. 8).

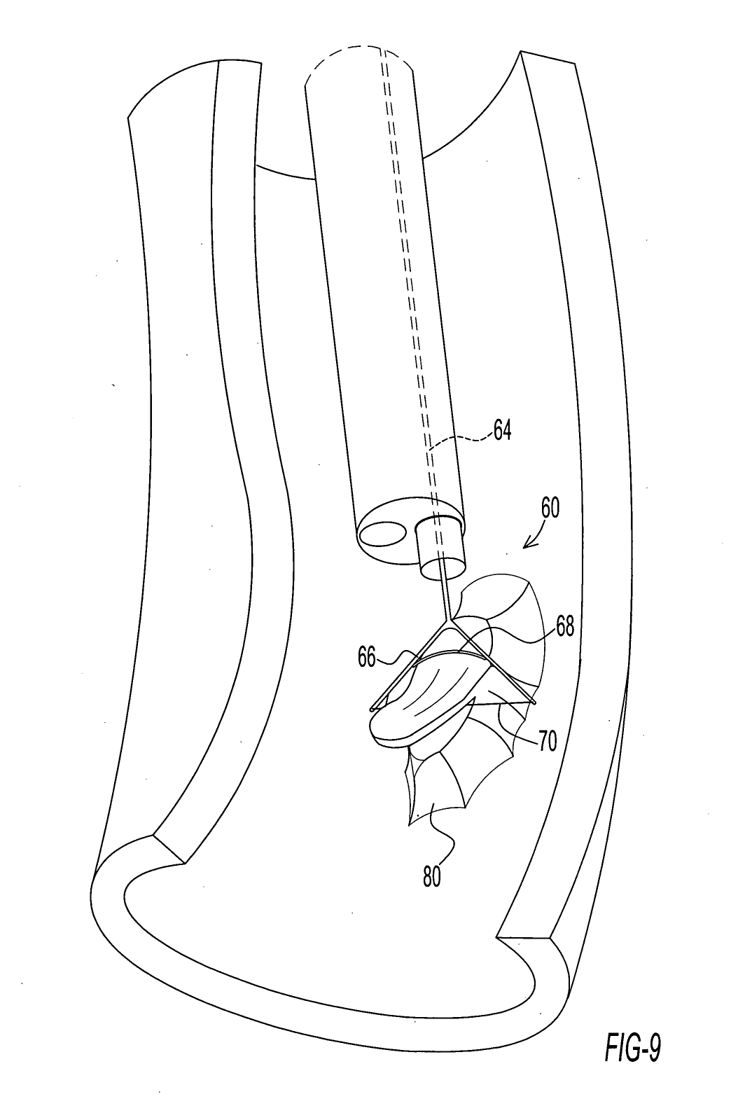

As illustrated in FIG. 9, when the cutting assembly 60 is in its fully open position ready for tissue resection, it is brought to the distal end of the target tissue 80, pressed into the tissue and pulled along the surgical site while activating the cautery. This action causes thin slicing through the tissue 80, such as a tumor, in the same fashion as a cheese cutter slicing through cheese, thereby forming webs or sheets of separated tissue. The cutting depth may be judged by the operator. An additional insulated wire 73 may be connected to both rigid segments 66, the legs of the triangle, at a fixed distance from the cutting wire 70. As demonstrated in FIG. 8, the connection of an insulated wire 73 could be formed proximate to where the spring biased arch is connected to the rigid legs. This second wire may serve as a gauge for judging the cutting depth as in the first embodiment. The device could be manufactured with or without this insulation wire; oftentimes physicians would like to use their own judgment concerning the depth of the cut. Furthermore, the lesion may not be the same thickness in all its various parts, and may require different depths of cutting.

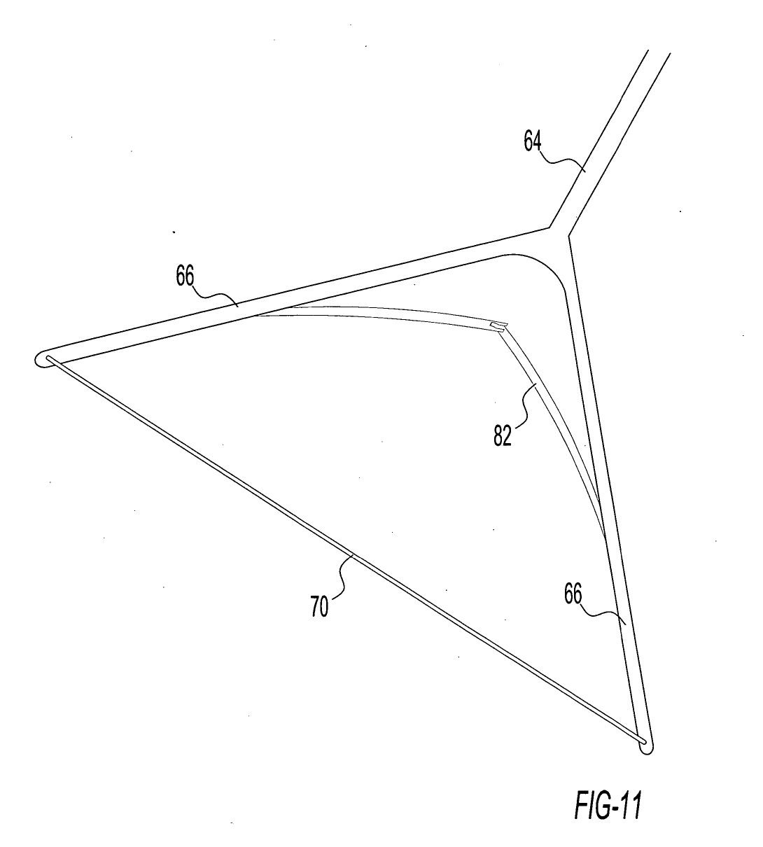

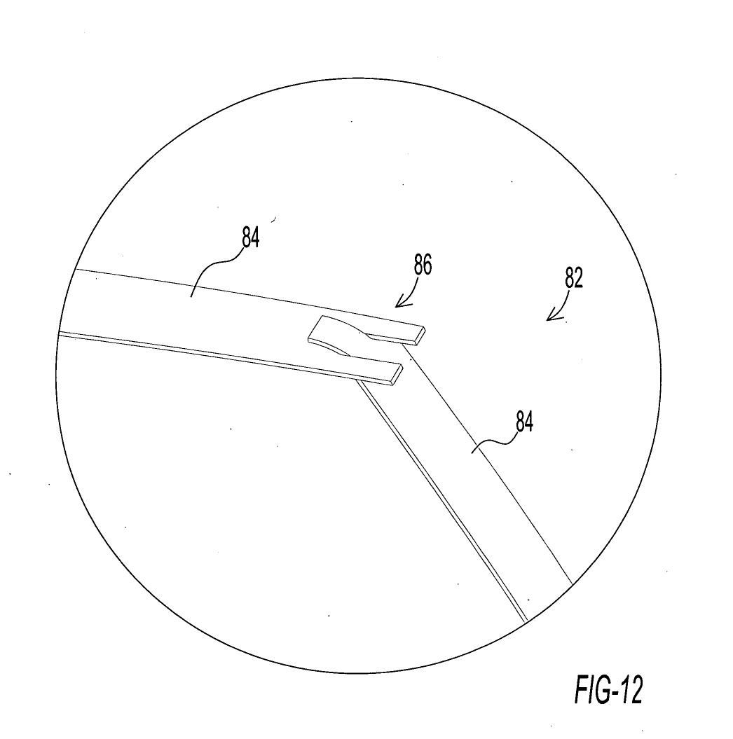

FIGS. 11-12 illustrate an alternative cutting device of the invention with an alternative arch structure 82. The arch structure 68 is illustrated as a single piece. However, alternatively, the arch structure 82 might be multiple pieces coupled together to provide a spring bias to drive the segments 66 apart. Referring to FIG. 12, the arch structure or spring 82 might utilize two legs 84 that cooperate, such as with a tongue and groove relationship 86 to provide a spring bias. This configuration enhances the folding or collapsing of the distal cutting assembly into the elongate tube.

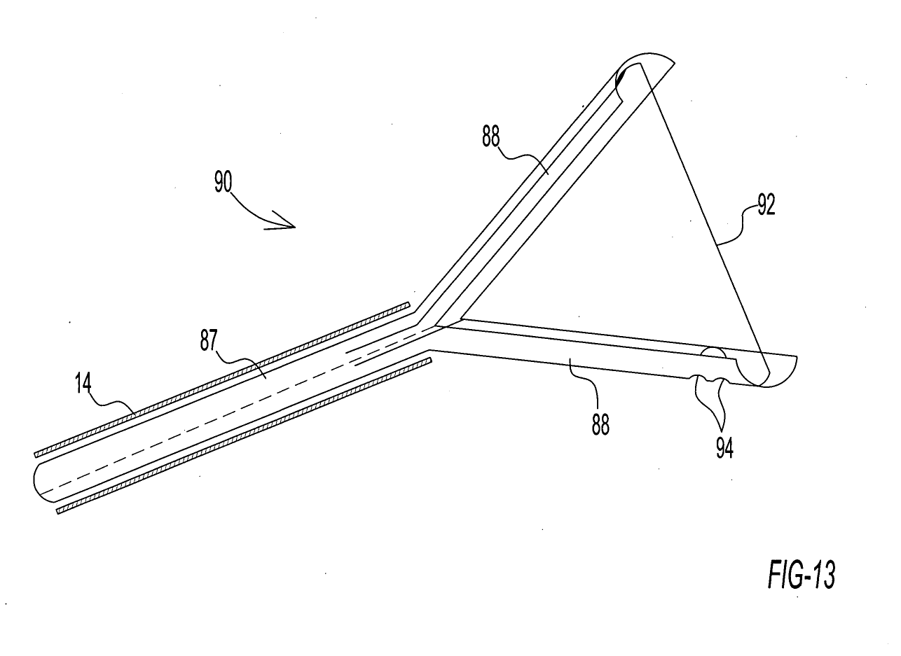

Another alternative embodiment of the cutting device is shown in FIGS. 13 and 14. The cutting device 90 is an electrocautery device similar to device 60, capable of being passed through the working channel of an endoscope. Like device 60, this iteration of the device is passed through an elongate tube, possesses a handle mechanism and a push rod all not shown in FIG. 13. However, in this embodiment the push bar comprises a hollow tube 87, which is split down the middle at its distal end into two longitudinally cut halves, comprising legs 88 of a wire holder (not separately designated) of the distal cutting assembly. The tubular push rod is made of a shape memory metal such as Nitinol. The inverted “V” shape that is created by cutting the tube as described above is baked into the shape memory metal such that when it is in an unconfined space, it takes on the shape of the inverted “V”. When it is stored in the pre-deployment configuration inside the elongate tube, the inverted “V” is closed, enabling storage of the distal assembly inside the elongate tube. The cutting wire 92, which may be a stainless steel monofilament, traverses through the tubular structure originating from its most proximal end, then runs down one leg 88 of the inverted “V”, forms a taut straight line at the base of the V when it is in its open position, and runs up the second leg 88 of the inverted V back into the tube 87. The proximal ends of the cutting wire traverse through the entire hollow push rod only to exit in the handle assembly, where they are operatively engaged with the electric connector. The reason for separating the wire from the shape memory material is that Nitinol and other shape memory materials are poor and unpredictable conductors of electricity. Therefore, by running the cutting wire through the legs of the inverted “V”, the operator is assured of consistent cutting action. The cutting wire may be affixed to the distal aspects of the inverted “V” legs by traversing through holes 94 or by crimping them onto the distal aspect of the legs. The legs are then covered by a nonconductive heat shrink material 96 as shown in FIG. 14, which serves to contain the cutting wire, and render the legs 88 non-conductive. FIGS. 13 and 14 show this iteration of the device in the open, deployed configuration, ready for the cutting operation. When the distal assembly is stored in the elongate tube, the legs of the inverted “V” come together; the cutting wire collapses and is situated in a folded fashion distal to the tips of the legs, and is thereby stored inside the elongate tube 14.

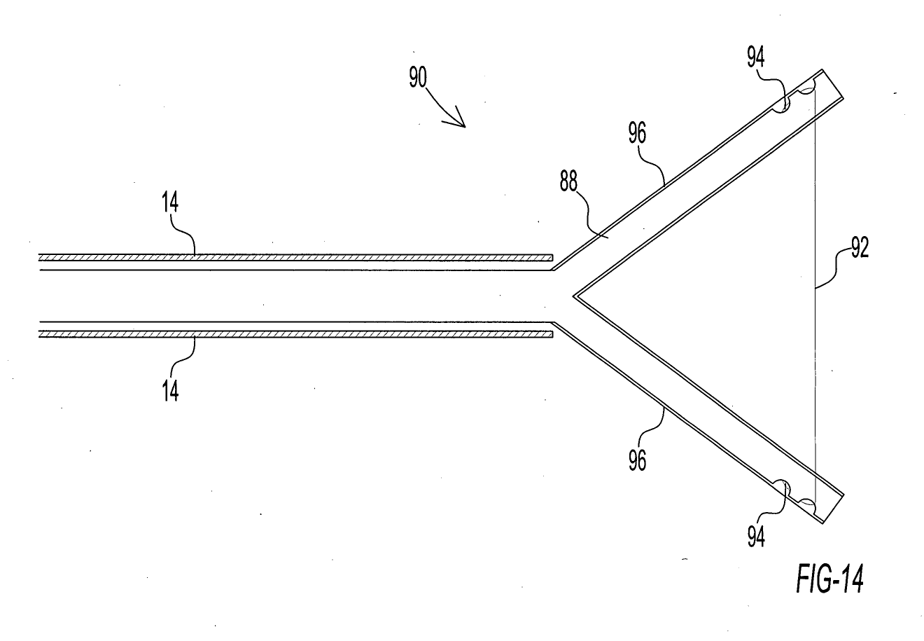

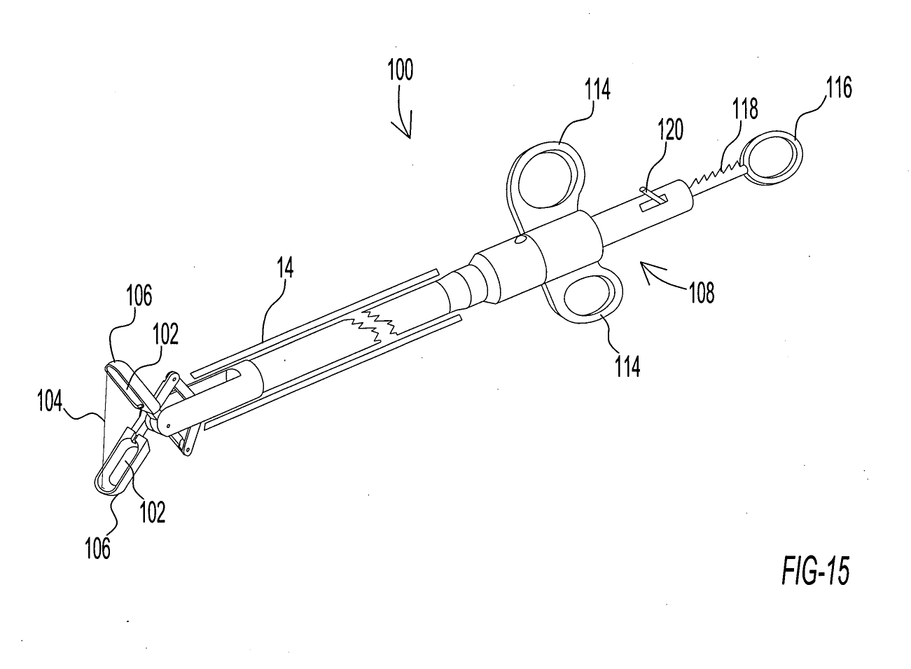

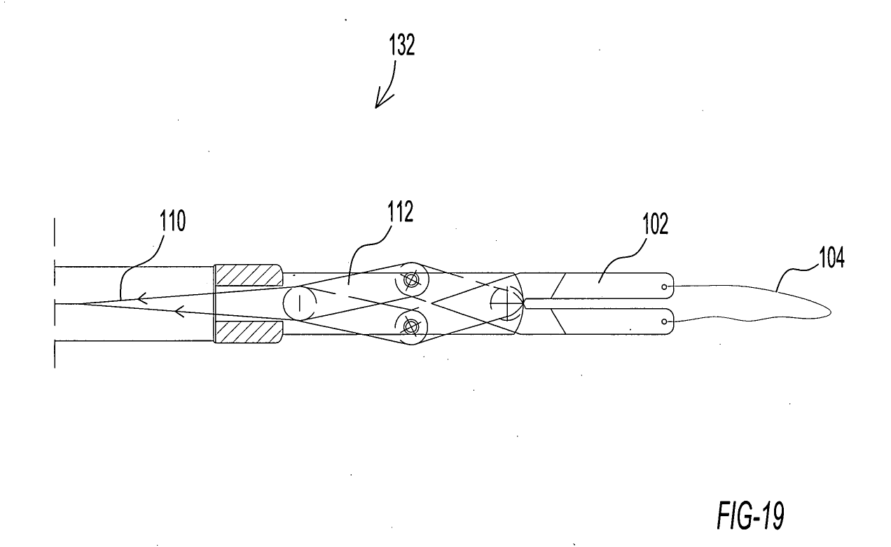

Yet another preferred embodiment of the device is illustrated in FIGS. 15-19. FIG. 15 demonstrates the entire device 100, whereby elongate tube 14 houses the cautery device for passage though the channel of the endoscope. A set of opposing jaws 102 are located at the distal end of the device. An electrically conductive wire 104 is held by and operatively connected to the distal tips 106 of the jaws 102. One ore more electrically conductive push-pull wires 110 shown in FIG. 19 are operably connected to links 112, which in turn move jaws 102 distally. Push pull wires 110 are also operatively connected to handle mechanism 108 proximally. In the embodiment illustrated in FIG. 15, handle assembly 108 includes finger rings 114 which serve to allow for traction while the thumb ring 116 is being pulled towards the operator. This action causes a pulling of the push-pull wires 110, which are proximally connected to thumb ring 116 and to the proximal jaw-links 112 distally. When thumb ring 116 is pulled, a ratcheting mechanism 118 coupled with locking mechanism 120 ensures locking of the wires 110 in the taut position, rendering the jaws 102 in their maximal open position, and the attached cutting wire 104 in its taut straight cutting configuration.

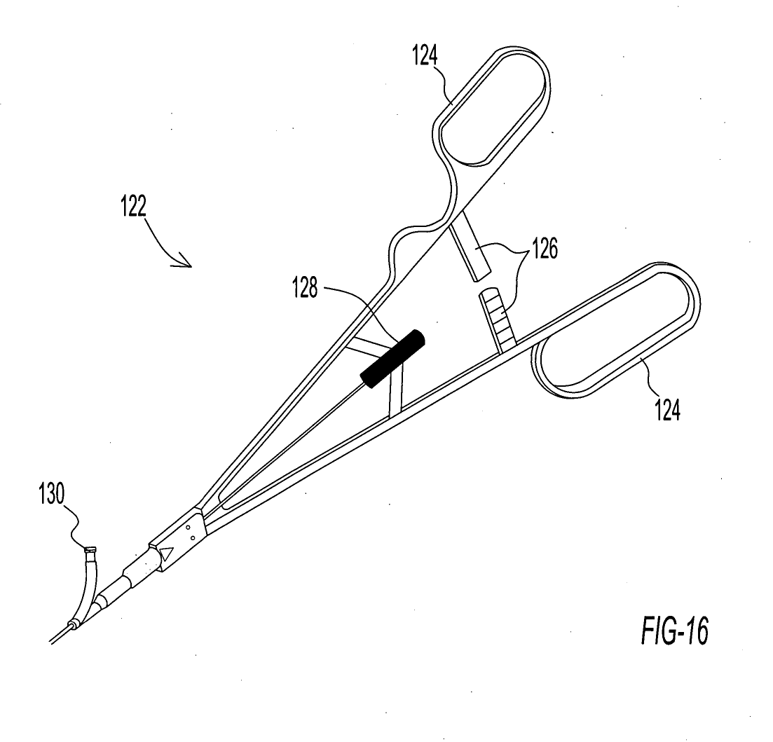

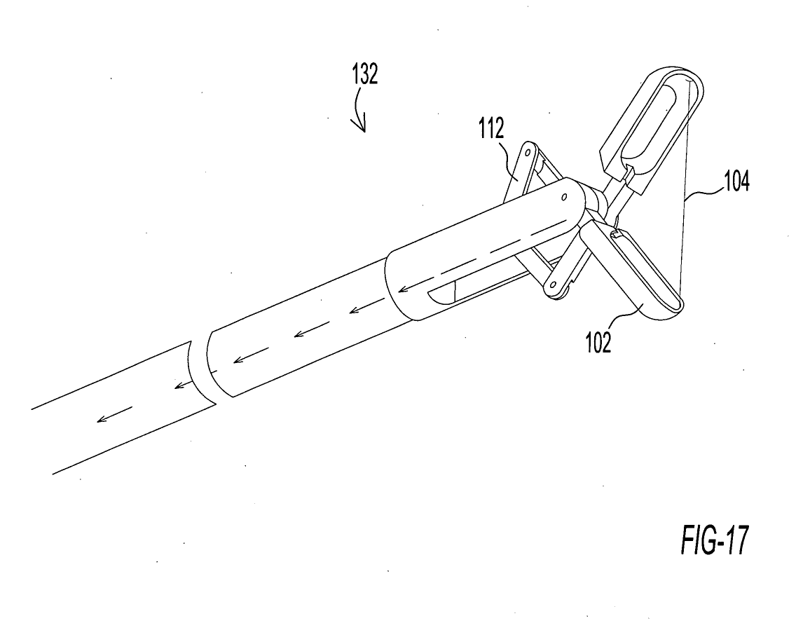

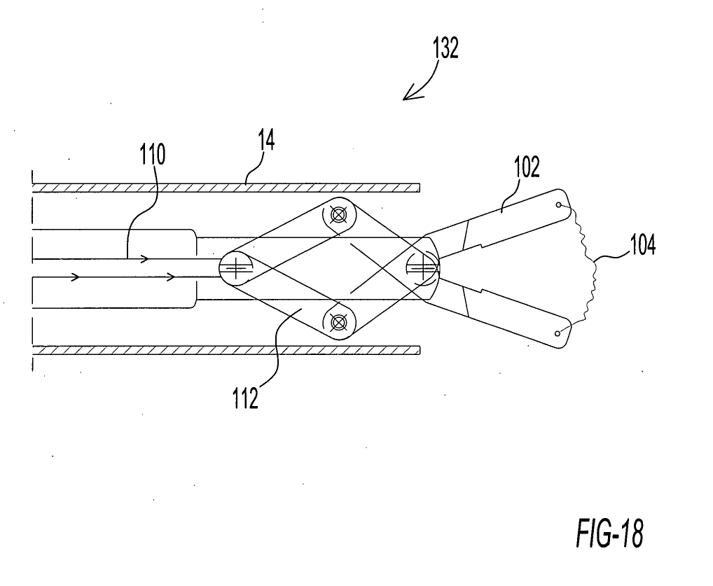

FIG. 16 illustrates another scissor handle mechanism 122. When the finger rings 124 are pulled open, the push-pull wire or wires 110 are pulled, causing the jaws 102 to open. The ratcheting mechanism 126 allows for continuous stable opening of the scissor handle, while the locking mechanism 128 locks the device in its operative open configuration ready for cutting. Electric connector 130 is coupled with push-pull wires 110, whereby when connected to an electric generator electricity flows through wires 110, into electrically conductive links 112, into jaws 102, and finally into cutting wire 104. FIG. 17 demonstrates the distal assembly 132 in its fully open cutting configuration. FIG. 18 demonstrates the distal assembly 132 in a partially open configuration, and FIG. 19, in a closed configuration. As in the case of device 60, the cutting wire is collapsed and folded, and is contained within the elongate tube 14 while in the pre-deployment configuration. When the device is prepared for use, the scissor handle 122 is ratcheted open to the maximum extent, and locked into position. The distal cutting assembly 132 is brought to the area to be resected, and while cautery is being activated is pulled across the lesion towards the operator, thus shaving a slice of thin tissue. The tissue is then retrieved and sent for pathologic examination.

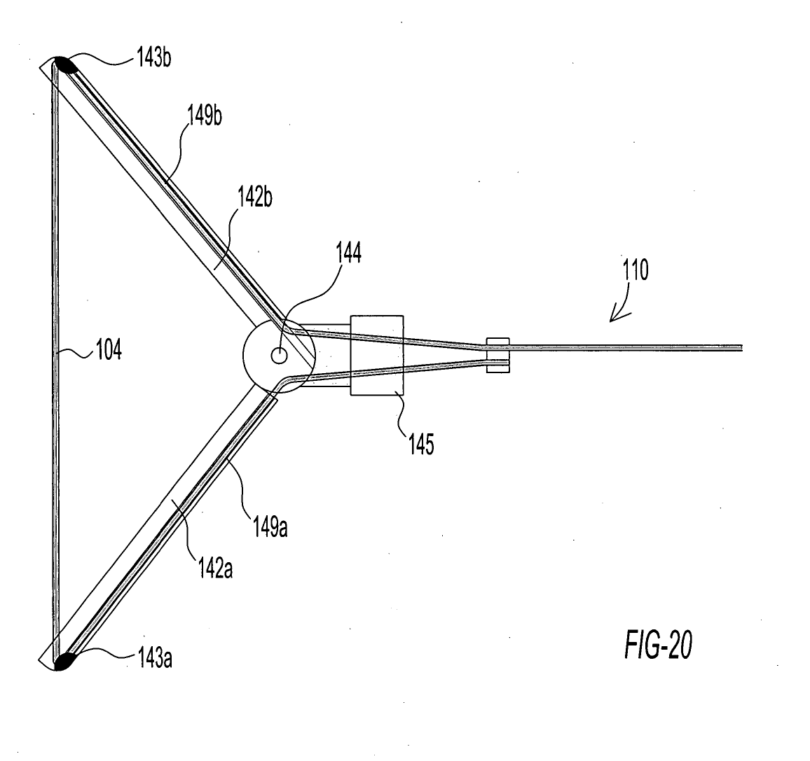

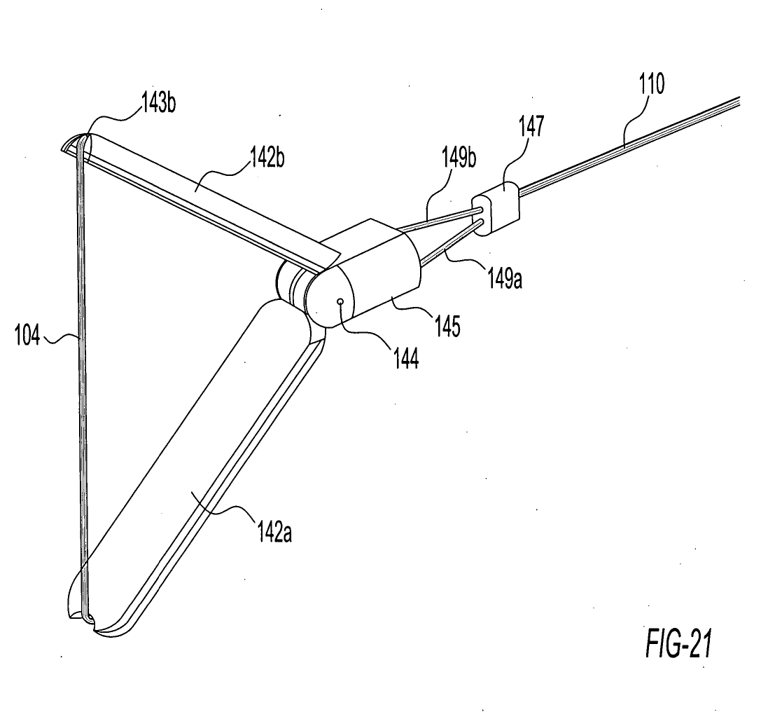

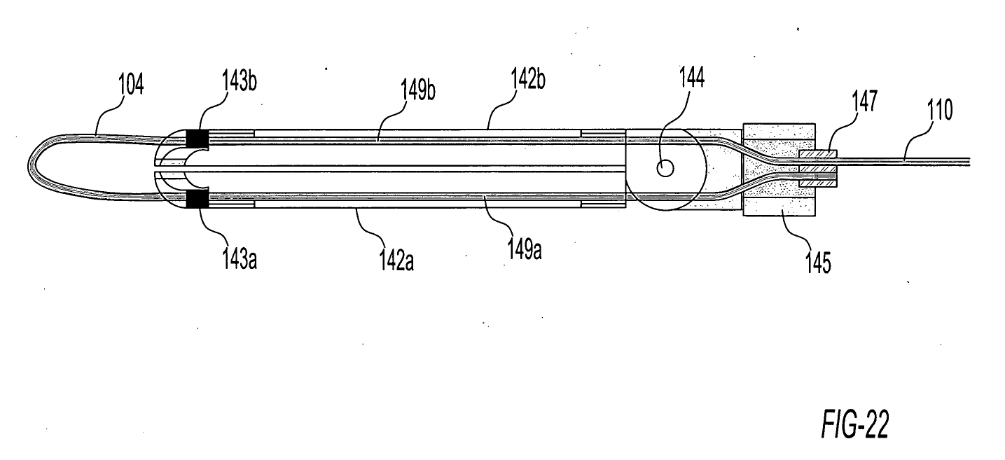

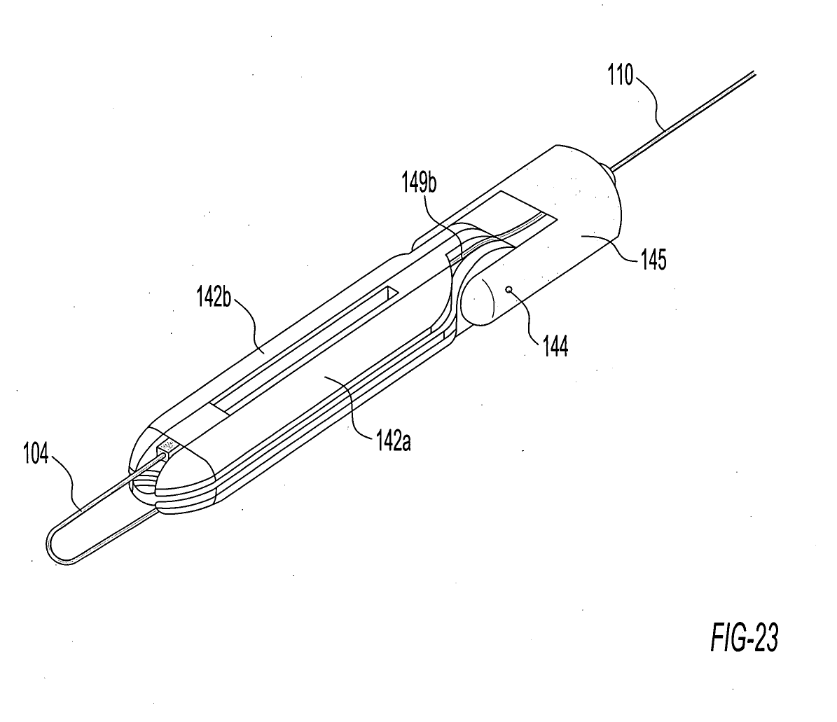

FIGS. 20-26 depict another tissue-shaving cutting and cauterizing device for use in an endoscopic procedure. FIG. 20 demonstrates an operative tip 141, at the distal end of the device, in an open, ready to cut position. A set of opposing jaws or arms 142a and 142b are located at the distal end of the device. Jaws 142a and 142b are hingedly secured to each other by a pivot pin 144. Pivot pin 144 is operatively connected to a pronged collar 145 which is permanently attached to an elongate instrument shaft such as tubing 14 and provides for a secure pivot point for jaws 142a and 142b. Jaws 142a and 142b are configured into right and left halves so that the distal tips align with one another in an open use configuration or a closed deployment or insertion configuration. An electrically conductive wire 104 is operatively connected to the distal tips of the jaws 142a and 142b, for instance, via insulating plugs 143a and 143b. This wire 104 is contiguous with push pull wires 110, which are proximally connected to a handle mechanism (not shown). FIG. 21 depicts the assembly from another angle. As cutting wire 104 is pulled proximally and tightened, jaws 142a and 142b are pulled open and the wire becomes taut. As cutting wire 104 is pushed, the jaws 142a and 142b close, and the cutting wire relaxes as shown in FIGS. 22 and 23.

Jaws or arms 142a and 142b constitute a holder member with wire 104 extending between spaced tips of jaws 142a and 142b in the use configuration (FIGS. 20 and 21). Jaws 142a and 142b and wire 104 form an operative tip that is introduced in a collapsed insertion configuration (FIGS. 22 and 23) through an endoscope working channel into a patient. At an operative site, instrument shaft or tubing 14 is pushed in the distal direction along the endoscope working channel so that jaws 142a and 142b emerge from the distal end of the working channel. Pusher wire 110 is then shifted in the distal direction to open jaws 142a and 142b and hold cutting and cauterizing wire 104 in a taut and straight configuration shown in FIGS. 20 and 21. The cutting and cauterizing instrument and endoscope are manipulated from outside the patient to place the straightened wire 104 into contact with a target tissue mass such as a sessile polyp or Barrett's esophagus formations. The cutting and cauterizing instrument is moved along the tissue mass to slice a thin layer, sheet or web of organic tissue off of the tissue mass. The process is repeated until the entire mass is removed. Because the shaving of the tissue is an iterative process, care can be easily taken to avoid a perforation of the organ wall (e.g., colon or esophagus).

Jaws or arms 142a and 142b define a Y- or V-shape and extend at an acute angle relative to one another in the use configuration (FIGS. 20 and 21). Jaws or arms 142a and 142b are pivoted away from one another to open the holder from the collapsed insertion configuration (FIGS. 22 and 23) to the spread-out use configuration (FIGS. 20 and 21) and to stretch the wire element 104 from a loose or flaccid loop (FIGS. 22 and 23) to a straight and taut line (FIGS. 20 and 21). This conformational change may be effectuated by simply pulling push rod or wire 110 in the proximal direction. A crimping element 147 enables a transfer of tensile forces to both jaws 142a and 142b, via wire segments 149a and 149b that extend from crimping element 147 along respective jaws or arms 142a and 142b.

As depicted in FIGS. 24-26, a jaw 150 utilizable in the cutting and cauterizing instrument of FIGS. 20-23 includes a lever arm 152, a pivot-pin retaining bearing 154, and a guide tube 156 which is traversed by a wire segment 149a or 149b (FIGS. 20 and 22). A notch or recess 158 is formed at a free end of arm 152 for turning the wire segment 149a or 149b into wire 104.

FIG. 27 shows a modification of the cutting and cauterizing instrument of FIGS. 20-23 where a helical spring member 160 is provided for biasing jaws 142a and 142b towards the open or use configuration. A lock (not shown) may provided on the handle or actuator assembly (not shown) for maintaining the jaws 142a and 142b in a closed or collapsed configuration during insertion of the instrument into the patient.

FIGS. 28-32 illustrate an endoscopic cutting and cauterizing instrument 161 including an elongate flexible tubular instrument shaft 162 insertable through a working channel 164 (FIG. 32) of an endoscope 166. The instrument also includes a holder member 168 provided at a distal end of the instrument shaft 162, and a cutting and cauterization wire element 170 connected at a distal end to a distal tip 172 of the holder member. In a use configuration of the holder member 168 and the wire element 170, the wire element extends taut in a straight line between distal tip 172 and a bend, joint or articulation 174 of the holder member, as shown in FIGS. 28, 31, and 32.

In the use configuration, holder member 168 has a V-shaped configuration with a pair of arms 176 and 178 extending at an acute angle a1 (FIG. 31) relative to one another. At its distal end, wire element 170 extends in a straight line from the one arm 176 of the holder member 168 to another arm 178 thereof. The arms 176 and 178 of the holder member are pivotably connected to one another at a bend, joint or articulation 180. Arm 178 is connected to shaft 162 via bend, joint or articulation 174.

In an insertion configuration illustrated in FIG. 29, arms 176 and 178 are disposed linearly exemplarily inside an outer tubular introduced sheath 182 that is in turn inserted inside the working channel 164 of the endoscope 166. After insertion of distal end portion of the endoscope 166 into a patient, an operative tip 184 of the cutting and cauterizing instrument 161 is ejected from the endoscope working channel 164. The operative tip 184, including arms 176 and 178 and the distal end portion or wire element 170, is then reconfigured from the linear insertion configuration (FIG. 29) to the bent or folded use configuration (FIGS. 28, 31, 32).

Arms 176 and 178 of holder member 168 are tubes that may incorporate a coiled spring member internally (represented by concatenated circles 186) that provides the holder member 168 with a spring bias tending to straighten arms 176 and 178 into the linear insertion configuration of FIG. 29. Arms 176 and 178 may be continuously formed with one another and with at least a distal end portion of instrument shaft 162. More particularly, arms 178 and 176 may be integrally formed with instrument shaft 162. This form of holder member 168 is one continuous catheter that has a small spring 186 inside it extending from the distal end to the most proximal end of arm 178. When in the pass-through configuration, the catheter is straight.

Upon the ejection of holder member 168 from working channel 164 of endoscope 166 (or from tubular introducer sheath 182, itself slidably inserted inside working channel 164), holder member 168 is folded first at joint, bend point, or articulation 180 to form arms 176 and 178 into a triangular or V-shaped configuration 188 shown in FIG. 30. This conformation change may be effectuated by pulling on the wire element 170, which extends along shaft 162 to a handle 189 (FIG. 28) at the proximal end of the instrument assembly. Subsequently, holder member 168 is folded at joint, bend point, or articulation 178 to fold V-shaped configuration 188 against tubular instrument shaft 162 to form the use configuration shown in FIGS. 28, 31, and 32. This conformation change may be effectuated by pulling on an auxiliary wire or thread element 190, which extends from joint, bend point, or articulation 180 through a hole 191 into outer tubular introducer sheath 182 and to handle 189.

FIG. 32 depicts use of the instrument assembly 161 to cut thin layers, sheets, or webs 192 of organic tissue from a sessile polyp or other tissue mass 194 along a wall 196 of an internal organ 198 such as the colon or esophagus. Current is conducted through wire 170 into the patient's tissues during a pulling of the operative tip 184 and particularly wire 170 through tissue mass 194 generally parallel to wall 196 and in a proximal direction towards a distal end face 200 of endoscope 166.

As illustrated in FIGS. 33, 34A-34D, and 35A-35C, a medical cutting and cauterizing device 202 comprises an elongate instrument shaft 204 insertable through a working channel 206 of an endoscope 208, a holder member 210 provided at a distal end of the instrument shaft, and a cutting and cauterization wire element 212 connected to the holder member. Wire element 212 may be made of tungsten. Wire element 212 may alternatively be made of a semi-rigid stainless steel, and cut through tissue without cauterization action. Wire element 212 extends between spaced points of holder 210 member in a use configuration of the holder member and the wire element. In this embodiment, the use configuration of holder member 210 and wire element 212 is identical to the insertion configuration, except for the location of the cutting and cauterizing device 202 relative to endoscope 208 and particularly relative to a leading or distal end face 214 thereof. This is to say that holder member 210 and wire element 212 are substantially rigid components connected to one another in a fixed configuration.

Holder member 210 typically but not necessarily has a V-shaped configuration with a pair of arms 216 and 218 extending at an acute angle a2 relative to one another. Wire element 212 extends along a circular arc from a tip or free end of arm 216 of the holder member to a tip or free end of arm 218. Holder member 210 and wire element 212 comprise an operative tip 219 of instrument 202 and lie in a plane oriented perpendicularly to instrument shaft 204, at least during an insertion or deployment procedure. More particularly, holder member 210 and wire element 212 are disposed along and flush against distal end face 214 of endoscope 208 and inserted into a patient while riding on the distal end face of the endoscope insertion member 220.

As shown in FIGS. 34A-34D, holder member 210 may be removably connectable to the end of instrument shaft 204. At the commencement of an endoscopic procedure, instrument shaft 204 is inserted into working channel 206 of endoscope 208 from the proximal end thereof. As shown in FIG. 34A, instrument shaft 204 is provided at a distal end with an internally threaded recess 222, while holder member 210 includes a stem 224 provided with an externally threaded pin 226. As indicated by an arrow 228 in FIG. 34B, holder member 210 is screwed onto the distal end of instrument shaft 204 as it protrudes from the distal end of endoscope working channel 206 prior to and in preparation of insertion into the patient. After formation of this connection, instrument shaft 204 is pulled in the proximal direction, as indicated by an arrow 230 in FIG. 34C, until holder member 210 and wire element 212 are snug against the leading or distal end face 214 of the endoscope insertion member 220, as shown in FIG. 34D. Holder member 210 and wire element 212 are dimensioned so that the wire element is seated along a periphery or rim 232 of front or distal end face 214 and so that arms 216 and 218 miss or avoid various working elements on distal end face 214, including, for instance, an illumination outlet 234, a lens 236, working channels 238, an irrigation fluid outlet port 240, air channel etc. (FIG. 34D).

Endoscope insertion member 220 is inserted into the patient with the operative tip 219 (holder member 210 and cauterizing wire element 212) engaging or snugly touching the distal end face 214 of the endoscope 208, as shown in FIGS. 34D and 35A. After the scope has reached a diagnostic or surgical site inside the patient, for instance, a tissue mass 242 (FIG. 35A) such as in Barrett's esophagus with a dysplastic growth, shaft 204 is pushed in the distal direction along the endoscope working channel 206 so that operative tip 219 is separated from distal end face 214 of the endoscope insertion member 220, as shown in FIGS. 34C and 35B. Then, instrument 202 and endoscope 208 are manipulated form outside the patient to bring wire element 214 into engagement with tissue mass 242. Wire element 212 is drawn into and along tissue mass 242 to remove a thin layer or web 244 of the tissue, as shown in FIG. 34B. During the motion of wire element 212 through tissue mass 242, electrical current is conducted into the wire element to facilitate a cutting and cauterizing of the tissue. The drawing of wire element 212 into and along the tissue mass 242 may then include pulling the holder member 210 via shaft 204 from outside the patient to draw the wire element towards a distal end face of the endoscope. Alternatively, the entire scope with shaft 204 and holder member 210 entrained thereto may be moved in the proximal direction. In certain cases, the motion may be that of a pushing away rather than pulling of either the endoscope, the cutting device or both. After the separation of tissue layer or web 244, as shown in FIG. 35C, the process may be repeated until the entire undesirable tissue mass 242 is removed from organ wall 245. A retrieval net or other device (not shown) may be used to remove the separated tissue slices or webs 244 from the patient.

As depicted in FIGS. 36A and 36B, a cutting and cauterizing instrument or device 246 as described above with reference to FIGS. 33-35C may be provided with a joint or articulation 248 that enables a user to pivot an operative tip 250 from a transverse orientation (FIG. 36A) relative to an instrument shaft 252 to a parallel or longitudinal orientation (FIG. 36B) relative to the instrument shaft. In the transverse orientation, the operative tip 250, including a Y-shaped holder 254 with a pair of arms 256 and 258 and an arcuate wire element 260 extending between the arms, is disposable in contact with a front or distal end face of an endoscope (not shown). In the parallel orientation, the operative tip 250 extends in a plane (plane of drawing FIG. 36B) that is parallel to a longitudinal axis 262 of instrument shaft 252. Operative tip 250, including holder 254 and a stem piece 264, may be spring biased towards the transverse orientation. Pivoting of the operative tip 250 to the parallel orientation is effectuated, for instance, by sliding shaft 252 axially relative to a surrounding sheath 266. As joint or articulation 248 is moved into sheath 266, stem piece 264 assumes a collinear relationship with shaft 252. In another embodiment, one or more wires or rods (not shown) may extend along shaft 252 to a distal end of stem piece 264 for exerting a torque thereon. The wires or rods may be alternately pushed or pulled, to change the orientation of the operative tip 219 from transverse to parallel and back again.

A metal collet 268 may be provided at the distal end of sheath 266 to facilitate the transformation from the transverse orientation of FIG. 7A to the parallel orientation of FIG. 7B. When the endoscopist is ready to pull the endoscope 208 out of the patient, operative tip 219 may stay in the parallel position, just as a cauterization snare with a pouch and a polyp may be pulled out without creating a problem.

As depicted in FIGS. 37A through 37C, a cutting and cauterizing instrument or device 270 as described above with reference to FIGS. 33-35C may be provided with a joint or articulation 272 that enables a user to pivot an operative tip 274 from a transverse orientation (FIG. 37A) relative to an instrument shaft 276 to a parallel or longitudinal orientation (FIG. 37B) relative to the instrument shaft. In the transverse orientation, the operative tip 274, including a Y-shaped holder 278 with a pair of arms 280 and 282 and an arcuate wire element 284 extending between the arms, is disposable in contact with a front or distal end face of an endoscope (not shown). In the parallel orientation, the operative tip 274 extends in a plane (plane of drawing FIG. 37B) that is parallel to a longitudinal axis 286 of instrument shaft 276. Operative tip 274, including holder 278 and a stem piece 288, are maintained in either the transverse orientation (FIG. 37A) or the parallel or straightened orientation by an elongate elastic member 290 that extends through a longitudinal bore or channel (not separately depicted) in instrument shaft 276 and through a longitudinal bore or channel (not shown) in stem piece 288. Pivoting of the operative tip 274 to the straightened orientation is effectuated, for instance, by sliding shaft 276 axially relative to a surrounding sheath 292. As joint or articulation 272 is moved into sheath 292, stem piece 288 assumes a collinear relationship with shaft 276.

A distal end (not separated labeled) of instrument shaft 276 is provided with a transverse slot 294, while a proximal end of stem piece 288 is optionally provided with a transverse slot 296. Slots 294 and 296 accommodate and facilitate a shifting of elastic member 290 during a rotation of stem piece 288 from the transverse orientation to the parallel or straightened orientation.

Wire element 212 may be constructed as a semicircle, or ¾ of a circle, and even as a straight cutting wire. The arcuate shape of wire element 212 is optimal for working in the esophagus, which has a rather restricted, circular lumen. The lesion may be removed by bringing the instrument below the lesion, and slowly burning off thin layers of tissue. The process may be quite controlled as to depth and breath. Clean margins are now created, no gaps need occur, and the muscularis need never be invaded and breached.

The EMR procedure sometimes requires injection of saline to raise the area for creating a buffer, or for injection of dye to mark the spot. It is therefore advantageous to provide either a double lumen that would house the shaft and instrument 202 in one lumen and a needle in another, or one lumen that would house them both. A snare with a web member may also be included in the assembly, preferably in a second or third lumen if the web member is to include a tether.

Instrument 202, as well as the other wire-implemented cutting instruments disclosed herein, is quite advantageous for EMR of sessile colonic polyps. The procedure may be performed as described above. The endoscope 208 can be bent 360 degrees in a circular motion, allowing for good contact and control. However, it may become desirable at a certain point, especially in the case of colonic polyps located in and around a bend in the colon or other lesions that are difficult to reach, to have the operative tip 219 device convert from a perpendicular (transverse) to a vertical (parallel) position, as described hereinabove with reference to FIGS. 36A and 36B. During insertion of an endoscope with instrument 202, the operative tip 219 cannot be disposed in a parallel or vertical position as such a position will block the endoscopist's view and interfere with insertion of the endoscope. Instead, the operative tip 219 must lie flat in an orientation perpendicular to the working channel and the instrument shaft, along the end face of the endoscope.

As it is important that the operative tip 219 does not move out of place while the endoscope 108, 208 is being inserted into the patient, stems 124, 224 and posts or arms 116, 216, 218 are constructed such that there is a snug fit into the working channel of the distal end of the instrument shaft, such as stem 264. This is accomplished by making this distal instrument shaft portion larger that the main body of the shaft. In addition, by pulling the device 102, 202 until the operative tip 119, 219 is in snug engagement with the endoscope tip, there is no opportunity for the distal assembly to be displaced during the insertion procedure.

A device may be offered with one shaft with handle, and several working-end assemblies to be attached as per the requirement of the surgeon. The handle assembly includes a plug for cautery, which is activated when surgery is performed. This idea is novel in the art of interventional flexible endoscopy: there are no devices at present that may be operated through the working channel of a flexible endoscope, which possess a substantially rigid end-working assembly that is larger than the working channel. This invention enables the use of such a larger end-assembly by passing the shaft of the instrument into the endoscope's working channel, and then attaching the end assembly distally prior to insertion into the patient. The end assembly must be “invisible” to the endoscopist until he or she are ready to use it. At that point the device is pushed forward, comes into view, and may be utilized for the operation.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A medical device comprising:
at least one elongate instrument shaft insertable through a working channel of an endoscope;
a holder member provided at a distal end of said instrument shaft; and
a cutting wire element connected to said holder member,
said wire element extending between spaced points of said holder member in a use configuration of said holder member and said wire element.

2. The medical device defined in claim 1 wherein said holder member includes, in said use configuration, a pair of arms extending at an angle relative to one another, said wire element extending from one of said arms to another of said arms.

3. The medical device defined in claim 2 wherein said arms are pivotably connected to one another.

4. The medical device defined in claim 3 wherein said arms are each connected at a proximal end to said instrument shaft.

5. The medical device defined in claim 4 wherein said arms are spring biased towards an opened configuration. The medical device defined in claim 3 wherein said arms are connected in series to one another and to said instrument shaft, said one of said arms being connected at one end to said instrument shaft and at an opposite end to said another of said arms.

6. The medical device defined in claim 2 wherein said arms are fixedly secured to one another, said arms and said wire element being disposable in a plane oriented perpendicularly to said instrument shaft.

7. The medical device defined in claim 6 wherein said holder element is disposable along a distal end face of an endoscope member.

8. The medical device defined in claim 7 wherein said holder element is removably connected to said instrument shaft.

9. The medical device defined in claim 6 wherein said wire element is substantially rigid or semi-rigid and has an arcuate form.

10. The medical device defined in claim 2 wherein said wire element originates from a proximal end of said instrument shaft, extends along said instrument shaft and said one of said arms, and forms a straight line from said one of said arms to said another of said arms in said use configuration.

11. The medical device defined in claim 1 wherein said wire element is made of an electrically conductive material for cauterizing organic tissues during a cutting operation.

12. A medical method comprising:
providing a medical instrument including an elongate wire element coupled to a holder member;
introducing at least a portion of said instrument including said wire element and said holder member into a patient via an endoscope;
thereafter placing said wire element into engagement with a patient's organic tissue at a surgical site; and
moving said wire element into and along said tissue to remove a thin layer or web of said tissue.

13. The method defined in claim 12 wherein said wire element is made of an electrically conductive material, further comprising conducting electrical current into said wire element, during the moving of said wire element into and along said tissue, to facilitate a cutting and cauterizing of said tissue at said surgical site.

14. The method defined in claim 12 wherein said wire element extends between two points on said holder member, the moving of said wire element into and along said tissue including shifting said holder member from outside the patient to move said wire element relative to a distal end face of said endoscope.

15. The method defined in claim 12 wherein said holder member includes a pair of arms pivotably connected to one another, further comprising opening said arms from a collapsed configuration to an opened use configuration upon an ejection of said holder member from a distal end of a working channel of said endoscope, the opening of said arms including a pivoting of said arms relative to one another.

16. The method defined in claim 12 wherein said holder member includes a pair of arms connected in series to one another and to said instrument shaft, one of said arms being connected at one end to said instrument shaft and at an opposite end to another of said arms, further comprising bending or folding said arms relative to one another to form a use configuration upon an ejection of said holder member in a straightened configuration from a distal end of a working channel of said endoscope, the bending or folding of said arms including a pivoting of said arms relative to one another.

17. The method defined in claim 12, further comprising disposing said holder member and said wire element along a distal end face of an insertion member of said endoscope, the introducing of said wire element and said holder member into the patient including inserting said insertion member into the patient with said wire element and said holder member disposed along said distal end face of the endoscope insertion member.

18. The method defined in claim 12, further comprising inserting a shaft of said medical instrument into a working channel of said endoscope and then attaching said holder member to said instrument shaft, the disposing of said holder member and said wire element along said distal end face being performed subsequently to the attaching of said holder member to said instrument shaft.

19. A medical device comprising:
one or more elongate instrument shafts insertable through a working channel of an endoscope;
two arms pivotably connected to a distal end of said one or more instrument shafts, said arms being spring biased to open from a collapsed insertion configuration to an open use configuration in which said arms extend at an angle relative to one another; and
a wire element extending between spaced points of said arms in said open use configuration of said arms.

20. The medical device defined in claim 19 wherein said arms are made of an inherently spring biased material tending to pivot said arms into said open use configuration.

21. The medical device defined in claim 19, further comprising a spring element operatively connected to said arms.

22. A medical device comprising:
at least one elongate instrument shaft insertable through a working channel of an endoscope;
a holder member provided at a distal end of said instrument shaft, said holder member comprising at least a first rigid arm segment and a second rigid arm segments connected in series with one another and said instrument shaft, said holder member including a first joint connecting said first arm segment to said shaft and a second joint connecting said second arm segment to said first arm segment at an end thereof opposite said first joint; and
a cutting wire element extending freely of said first arm segment and said second arm segment, from said first joint to a distal end of said second arm segment.

23. The medical device defined in claim 22, further comprising a pull thread or wire operatively connected to said second joint for bending said first arm segment relative to said shaft at said first joint, said wire exerting a force on said distal end of said second arm segment to bend said second arm segment relative to said first arm segment at said second joint.

24. The medical device defined in claim 22 wherein said cutting wire is made of electrically conductive material and extends along said shaft toward a proximal end of said shaft, for connecting to a source of electrical current.

25. A medical device comprising:
at least one elongate instrument shaft insertable through a working channel of an endoscope;
a holder member provided at a distal end of said instrument shaft, said holder member comprising a pair of substantially rigid arms connected to one another at said instrument shaft; and
a substantially rigid or semi-rigid cutting wire element connected to free ends of said arms, said wire element extending freely in an arc between said ends of said arms.

26. The medical device defined in claim 25 wherein said arms are disposable in a plane oriented perpendicularly to said instrument shaft.

27. The medical device defined in claim 25 wherein said holder element is removably connected to said instrument shaft.

28. The medical device defined in claims 27 wherein said instrument shaft includes a pivotable stem section, said arms being rigidly connected to said stem section.
